(19) United States
(12) Patent Application Publication
(10) Pub. No.: US 2023/0240546 A1
(43) Pub. Date: Aug. 3, 2023

(54) FITNESS MONITORING AND TRACKING DEVICE

(71) Applicant: ORB Innovations Ltd., London (GB)

(72) Inventors: Robert Paterson, London (GB); Thomas Quinn, London (GB)

(21) Appl. No.: 18/008,221

(22) PCT Filed: May 28, 2021
(86) PCT No.: PCT/GB2021/051317

§ 371 Date: Dec. 5, 2022

(30) Foreign Application Priority Data
Jun. 5, 2020 (GB) 2008477.8

(51) Int. Cl. A61B 5/024; A63B 71/08

(52) U.S. Cl. CPC A61B 5/02438; A63B 71/085; G16H 40/67

(57) ABSTRACT

A mouthpiece for a fitness tracking and/or monitoring system, the mouthpiece comprising a retainer member shaped and configured to be worn, in use, within a user's mouth, the retainer member being formed of at least two layers of elastomeric material having a physiological sensor therebetween.

### INTRODUCTION

This invention relates to a wearable fitness tracking and/or monitoring device for monitoring fitness and other physiological metrics, such as heart rate (and/or other cardio-respiratory signals) of a user, for use in, for example, sports and other activities.

### BACKGROUND OF THE INVENTION

Fitness tracking devices are well known and can be used to monitor a user's fitness-related metrics to provide feedback regarding their fitness and/or performance. Indeed, there is an ongoing desire to enable, for example, an athlete's fitness, fatigue and effort to be monitored during training and matches in order to provide key performance insights and reduce the risk of injury.

For example, heart rate monitors are known for use in measuring heart rate (and/or other cardio-respiratory signals) for applications such as health monitoring, sports training and fitness. Conventional devices capable of constantly monitoring a user's heart rate (and/or other cardio-respiratory signals) have, until recently, been rather cumbersome. For example, heart rate monitors often use electrocardiogram (EKG) signals, which require electrodes or a strap placed around the user's chest.

A less cumbersome technique for monitoring heart rate is based on pulse oximetry using a photoplethysmograph sensor. A photoplethysmograph sensor nonOinvasively measures the absorption of light passing through a user's tissue, e.g. a finger or ear lobe, to determine the oxygen saturation level of arterial blood and heart rate. The resulting signal is referred to as a photoplethysmograph (PPG), and the PPG signal can be analysed to determine, among other things, the heart rate (and/or other cardio-respiratory signals) of the person to which the PPG sensor is attached.

Conventionally, pulse oximetry has required the user to remain relatively motionless to ensure that a good PPG signal is obtained. When a PPG sensor is used with a person that is in motion, displacement of the sensor and motion artifacts or noise in the PPG signal can result in inaccuracies in the estimated heart rate (and/or other cardio-respiratory signals) of the user.

U.S. Pat. No. 10,456,053 describes a wrist worn heart rate monitor including a PPG sensor and an inertial sensor, wherein signals from the inertial sensor are used to identify and remove noise from the PPG signals. An initial heart rate value is selected from a number of heart rate candidates that remain in the resulting PPG spectrum and is used to track the heart rate of the user. The PPG spectrum is monitored while tracking the heart rate (and/or other cardio-respiratory signals) to determine if the selected initial heart rate value is in error. The PPG spectrum is monitored and the heart rate value reset as required.

Wrist worn PPG sensors of this type are becoming increasingly prevalent within fitness tracking watches and the like. However, watches or other wrist-worn devices are often not practical (or permitted) when playing contact sports such as rugby, as they can cause injuries to the wearer or other players. Furthermore, wrist-worn fitness trackers may not necessarily be sufficiently accurate for highly effective fitness tracking, at least in some applications, even with software to remove noise from the monitoring signals.

Performance tracking in professional sport is currently achieved with wearables such as GPS trackers and chest-worn heart-rate monitors, and/or video analysis. However, such wearables are known to restrict movement, and they all measure what is happening to the athlete, rather than how their body is reacting, and all such wearables are located within clothing or on the skin (e.g. chest strap) which allows them to move around during use, leading to inaccurate sensor measurements.

Thus, it would be desirable to provide an accurate, user-worn fitness and/or performance tracking device, that is convenient and safe to wear during contact sports and/or over long periods of time, and which provides accurate physiological signals that can be used to monitor the user's fitness, performance and/or health, and aspects of the present invention seek to address at least one or more of these issues.

### SUMMARY OF THE INVENTION

In accordance with a first aspect of the present invention, there is provided a mouthpiece for a fitness tracking and/or monitoring system, the mouthpiece comprising a retainer member shaped and configured to be worn, in use, within a user's mouth, the retainer member being formed of at least two layers of elastomeric material having a physiological sensor therebetween.

In a preferred embodiment, the sensor may be configured to generate signals representative of the heart rate (and/or other cardio-respiratory signals) of a user. The sensor may, beneficially, comprise a photoplesythmograph sensor comprising a light source for, in use, shining a light on a region of the inside of a user's mouth and a sensor for receiving light reflected from said region of the inside of the user's mouth and generating electrical signals representative thereof.

At least one of the at least two layers of elastomeric material nearest the inner surface of the user's mouth, when in use, may be substantially transparent. At least one of the two layers of elastomeric material furthest from the inner surface of the user's mouth, when in use, may be substantially opaque. Thus, if the sensor is a photoplethysmograph (PPG) sensor, the inner layer allows light to pass through to the user's skin and light reflected back from the blood vessel(s) to pass back to the sensor, and the outer layer prevents ambient light entering the user's mouth from interfering with the accuracy of the sensor.

The elastomeric material is, beneficially, an elastomeric polymer such as Ethylene-Vinyl Acetate (EVA).

The sensor is beneficially located within said retainer member such that, in use, it is located adjacent the user's palate. The inventors have, surprisingly, discovered that the PPG signal that can be obtained from the user's palate is clearer and more stable than that which could be obtained from other regions of the user's mouth.

The sensor may beneficially be incorporated on a substrate, said substrate further incorporating a control module, a power supply unit and a memory module. The substrate (e.g. printed circuit board or PCB) may further incorporate a wireless communication module and antenna configured to wirelessly transmit data representative of signals generated by said sensor to a remote receiver. Thus, physiological data generated by the sensor can be collected in substantially real time whilst the mouthpiece is being worn.

In an exemplary embodiment, the sensor may be incorporated at one end of a first elongate flexible connector. A second elongate flexible connector may be provided or integrally formed at the other end of the first elongate flexible connector (substantially at right angles thereto), said second elongate flexible connector having incorporated at one end thereof at least one or more of a control module, a power supply unit, a memory module, a wireless communication module and a wireless communication antenna. The first and second elongate flexible connectors, together, may form a substantially T-shaped flexible connector. Thus, in an exemplary embodiment, a PPG sensor can be located the free end of the central ‘leg’ of the T-shaped flexible connector (so that it is located adjacent the user's palate, in use), and other sensors and electronic components can be located at the free ends of the ‘arms’ of the T-shaped flexible connector. Other sensors may include an accelerometer for measuring impact.

The retainer member may, in some embodiments, be formed of at least three layers of elastomeric material, with said sensor being located between first and second layers, nearest the inner surface of the user's mouth when in use, and a third layer formed over said second layer. Again, the sensor beneficially comprises a photoplethysmograph sensor, and said first and third layers are substantially transparent and said second layer is substantially opaque. Once again, the photoplethysmograph sensor may be provided on a flexible connector and located within the retainer member such that, in use, it is positioned adjacent the user's palate.

In accordance with a second aspect of the invention, there is provided a mouthpiece assembly comprising a mouthpiece substantially as described above, and a storage case for receiving said mouthpiece, when not in use, the mouthpiece comprising a receiving element of a wireless charging system and said storage case comprising a transmitting element of a wireless charging system and, wherein said transmitting element is positioned adjacent said receiving element when the mouthpiece is located within the storage case.

The receiving element may comprise a conductive coil printed or otherwise provided on a flexible printed circuit board located between said at least two layers of elastomeric material, and said transmitting element may comprise a plurality of overlapping conductive coils.

In accordance with a third aspect of the present invention, there is provided a fitness tracking and/or monitoring system comprising at least one mouthpiece substantially as described above, a receiver for receiving physiological data from the sensor of said at least one mouthpiece and an analytics platform for analysing said physiological data and providing an output indicative of fitness and/or performance of the or each user.

In accordance with a fourth aspect of the present invention, there is provided a method of manufacturing a mouthpiece for a fitness tracking and/or monitoring system, the method comprising forming a positive mould of or representative of a user's teeth and gum, forming a first layer of elastomeric material over said mould, securing a physiological sensor circuit to said first layer of elastomeric material, and forming a second layer of elastomeric material over said physiological sensor circuit.

As before, the first layer may be substantially transparent, said second layer may be substantially opaque, and said sensor circuit may comprise a light source, a photoplethysmograph sensor, and a wireless communications module for wirelessly transmitting data representative of signals generated by said sensor to a remote receiver, in use.

These and other aspects of the invention will become apparent from the following detailed description.

### BRIEF DESCRIPTION OF THE DRAWINGS

Embodiments of the present invention will now be described, by way of examples only, and with reference to the accompanying drawings, in which:

FIG. 1 is a schematic diagram of a fitness tracking system according to an exemplary embodiment of the present invention;

FIG. 2 is a schematic diagram illustrating principal components of a PCB for a mouthpiece according to an exemplary embodiment of the present invention;

FIG. 3 is a schematic cross-sectional view of a mouthpiece according to an exemplary embodiment of the invention;

FIG. 4 is an illustration of PPG sensor data obtained by a fitness tracking/monitoring system according to an exemplary embodiment of the present invention;

FIG. 5 is a plan view of a PCB for a mouthpiece according to an exemplary embodiment of the present invention;

FIG. 6 is a schematic perspective view of a mouthpiece and storage case of a fitness tracking/monitoring system according to an exemplary embodiment of the present invention; and

FIG. 7 is a flow diagram illustrating the functionality of embedded firmware in a mouthpiece according to an exemplary embodiment of the present invention.

### DETAILED DESCRIPTION

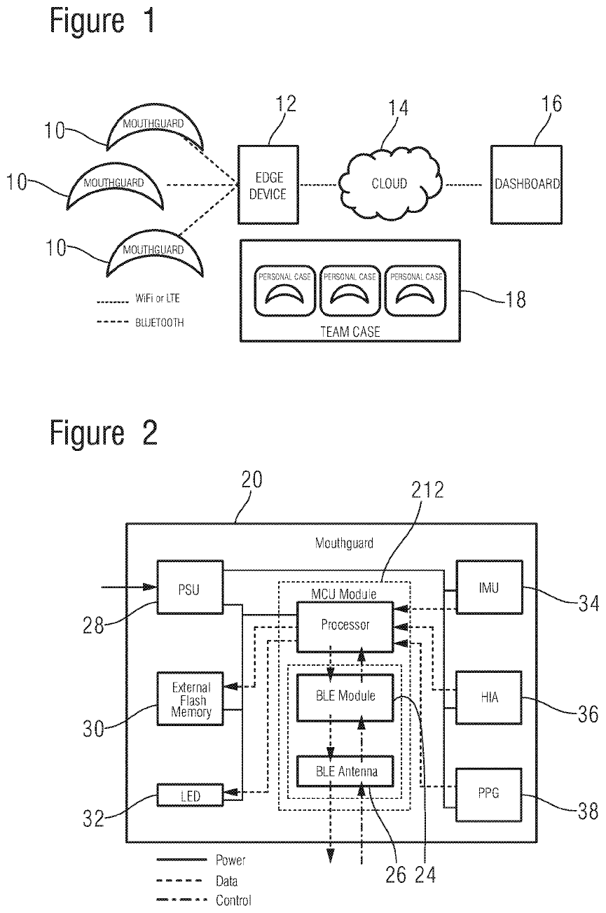

Referring to FIG. 1 of the drawings, a performance monitoring system according to an exemplary embodiment of the present invention comprises one or more health tracking devices 10 communicably coupled (or couplable) to an edge device 12. The edge device 12 is communicably coupled to a cloud processing/storage facility 14, which processes and analyses physiological data collected by the health tracking device(s) and outputs performance tracking data to a dashboard 16 on a remote web-based analytics platform accessed, for example, via an associated app. A storage case 18 may be provided, for storing and (optionally) charging one or more health tracking devices, when not in use.

The or each health tracking device 10 comprises a printed circuit board (PCB), incorporating one or more sensors, embedded in a mouthpiece such as a sports mouthguard, gum shield, orthodontic appliance, such as those used to alleviate or treat snoring or sleep apnoeia, retainer, etc. Such mouthpieces may be generic or they may be custom fitted to the user's mouth. The sensor(s) on the PCB generate physiological signals obtained whilst the mouthpiece is worn and wirelessly transmit the signals (or data representative thereof) to the edge device 12, which is essentially a receiver (or multiple receivers) configured to receive the data transmitted from the mouthpiece PCB. The primary purpose of the edge device 12 is to receive data from the mouthpiece, in use, and save the data locally and/or upload it to the cloud 14. Thus, it acts as a ‘gateway’ between the mouthpiece 10 and the analytics platform. The user's mouthpiece data is processed and analysed by a cloud (or local) computing platform 14, and a web-based dashboard 16 allows users to view their data and analytics in substantially real time on a personal computer, tablet, phone or any web browser.

Referring to FIG. 2 of the drawings, a PCB embedded in a mouthpiece according to an exemplary embodiment of the present invention may comprise a microcontroller (MCU) 212 having a Bluetooth Low Energy (BLE), or other wireless communication, module 24, and a BLE (or other wireless communication technology) antenna 26. The PCB 20 also carries a power supply unit (PSU) 28, an external flash memory module 30, an Led 32, an inertial measuring unit (IMU) 34, a high impact accelerometer (HIA) 36 and a pulse oximeter (PPG) sensor 38. The PSU 28 is electrically coupled, and supplies energy to, the MCU module 212, the IMU 34, the HIA 36, the PPG sensor 38, the LED 32 and the external flash memory module 30.

The IMU 34 may comprise a 3-axis magnetometer, a 3-axis accelerometer and a 3-axis gyroscope which, together in use, can be used to generate data representative of impacts on a player's head in terms of force, rotation and direction. The HIA 36 can be used to detect much greater impacts than the IMU. The pulse oximeter and heart rate sensor 38 is used to obtain a PPG which represents blood volume changes in the user's microvascular bed of tissue. The PPG sensor 38 works by shining a light source (from the LED 32) onto a region inside the user's mouth, e.g. the palate, and measuring the amplitude of green, red and infrared light reflected back to the sensor to obtain the PPG, in the ranges of 537 nm, 660 nm and 88 nm respectively. This allows for parameters to be collected that cannot typically be obtained in real time by prior art devices during sports, as set out in the table below.

TABLE 1
List of possible parameters/insights that can
obtained from embodiments of the invention

Anaerobic Threshold
Arterial Stiffness
Blood Pressure
Collision Count
Collision Load
Diastolic Amplitude
Dicrotic Notch Amplitude
Distance
Glucose
Heart Rate
Hydration
Injury Risk
Lactate Threshold
Onset Amplitude
Oxygen Saturation
Peak to Peak
Player Load
Power
Pulse Area
Pulse interval
Pulse Width
Rate Force
Reflection Index
Respiration Rate
Speed
Stiffness Index
Systolic Amplitude
Ventilatory Threshold
Work Rate

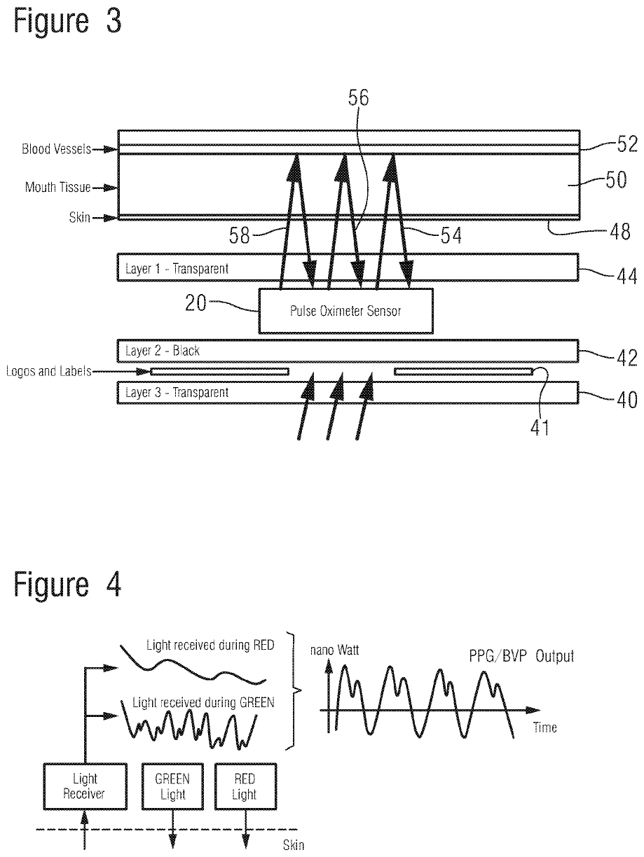

Referring to FIGS. 3 and 4 of the drawings, the mouthpiece comprises multiple thin layers of material. An outer, protective layer 40 is substantially transparent (i.e. allows light to pass therethrough. A middle layer 42 is black or of a very dark colour that substantially prevents light from passing therethrough. An inner layer (closest to the user's teeth and gum) 44 is substantially transparent (i.e. allows light to pass therethrough). Labels 41 carrying information and/or logos may be ‘sandwiched between the outer layer 40 and the middle, black layer 42. The PCB 20, including the LED 32 and the PPG sensor 38, is ‘sandwiched’ between the black layer 42 and the inner, transparent layer 44. Thus, any ambient light 46 entering the user's mouth is blocked by the black layer 42 and thus prevented from interfering with the PPG sensor 38. Light (between 10 nm and 3000 nm) from the LED 32 shines through the inner layer 44 onto the user's skin 48 and through the underlying mouth tissue 50 to the blood vessel(s) 52, and the reflected light returns to the PPG sensor 38, where the amplitudes of the reflected green 54, red 56 and infrared 58 light can be measured, and an example of data thus obtained is illustrated in FIG. 4 of the drawings.

The PSU 28 comprises, for example, a 38 mAh Lithium polymer battery which can be charged, for example, by a wireless charging arrangement using cooperative oscillating coils, with the transfer of energy occurring by means of magnetic induction between the coils.

A conventional mouthguard is formed of multiple layers of Ethylene-Vinyl Acetate (EVA) plastic, heated and vacuum formed, a layer at a time, over a positive mould of a user's teeth and gum. The layers of EVA are subsequently trimmed and polished (between formation of each layer) to achieve a smooth surface finish. A mouth guard or mouthpiece according to an embodiment of the invention may be manufactured as follows. In a first step, a positive mould of a user's teeth and gum is produced in any known manner. Next, a first, transparent layer 44 of EVA is heated and vacuum formed over the mould, and then trimmed and polished in a known manner. The PCB 20 is then stuck to the first layer 44, positioning the PPG sensor 38 under the ‘teeth’ onto the palate region. In this exemplary embodiment, the PPG signals are obtained from the user's palate, as it has surprisingly been discovered by the inventors to yield sufficiently clear and stable results. However, it will be appreciated that the PCB 20 could, in theory, be positioned in the mouthpiece such that PPG signals can be obtained from another part of the user's mouth. Next, a black layer 42 of EVA is heated and vacuum formed over the first layer 44 and

PCB 20, and then trimmed and polished. Labels/logos may be stuck to the black layer 42 if required. Finally, a third, transparent layer 40 of EVA is heated and vacuum formed over the black layer 42 and labels/logos (if present), and then trimmed and polished, as before, to form the finished device.

In an alternative exemplary method of manufacture, the second and third layers 40, 42 may be formed by, first, heating the black EVA layer 42 to its melting point, then applying any labels/logos thereto, and then finally slowly applying the third layer 40.

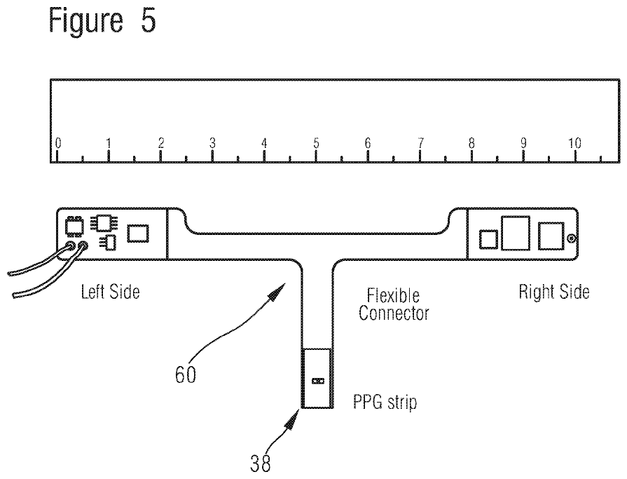

The PCB 20 may be configured to be ‘split’ into three separate sections, connected together by a flexible connector in the form of, for example, polyimide flexible PCB material. As shown in FIG. 5 of the drawings, the PCB 20 can be configured around a substantially ‘T-shaped’ flexible PCB 60, with the LED 32, external flash memory 30 and PSU 28 provided on a microchip at the end of one ‘arm’ of the flexible PCB, the HIA, IMU and MCU modules provided on a microchip at the end of the other ‘arm’ of the flexible PCB, and the PPG sensor provided at the end of the central ‘leg’ of the flexible PCB 60.

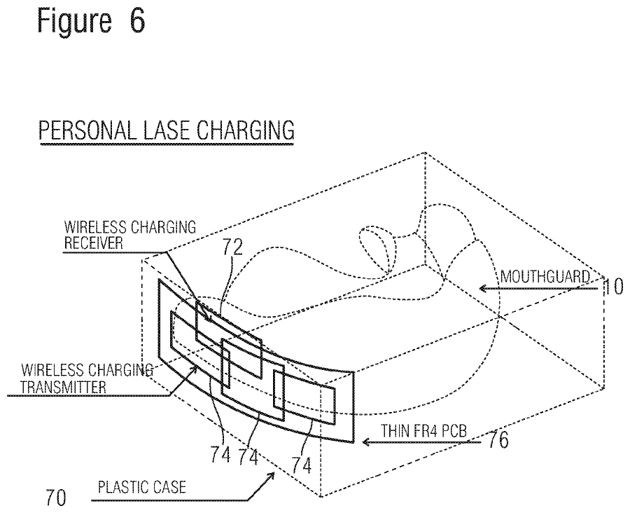

Charging of the PSU 28 may be achieved by means of wireless charging using two sets of conducting coils: one incorporated on the PCB 20 within the mouthpiece 10 and electrically connected to the Lithium polymer battery, and the other in a storage case (for example) and electrically connectable to a power source (e.g. by means of a USB (or other) charging cable (not shown). Referring to FIG. 6 of the drawings, a (e.g. plastic) storage case 70 houses a mouthpiece 10. A first coil 72 is provided within the mouthpiece 10, wherein the coil may be printed on the PCB 20 using copper traces, for example. In view of the unique shape and configuration of each custom fit mouthpiece, the storage case 70 may have a recess defining a negative mould of the user's teeth and gum, such that the respective mouthpiece 10 fits securely within the recess in the storage case 70. The recess may be coated with an antibacterial material if required. However, the external configuration of the storage case 70 is likely to be generic, e.g. of rectangular box-like configuration. Thus, the transmitting element of the wireless charging arrangement needs to be configured to cooperate with varying mouthpiece shapes. In order to address this issue, the transmitting element of the wireless charging arrangement comprises multiple overlapping coils 74 (to allow for misalignment in mouthpiece positioning) printed on a thin (slightly flexible) PCB 76 using copper traces, for example. A case may be provided, that is configured to ‘dock’ multiple individual storage cases or mouthpieces for simultaneous storage and/or charging.

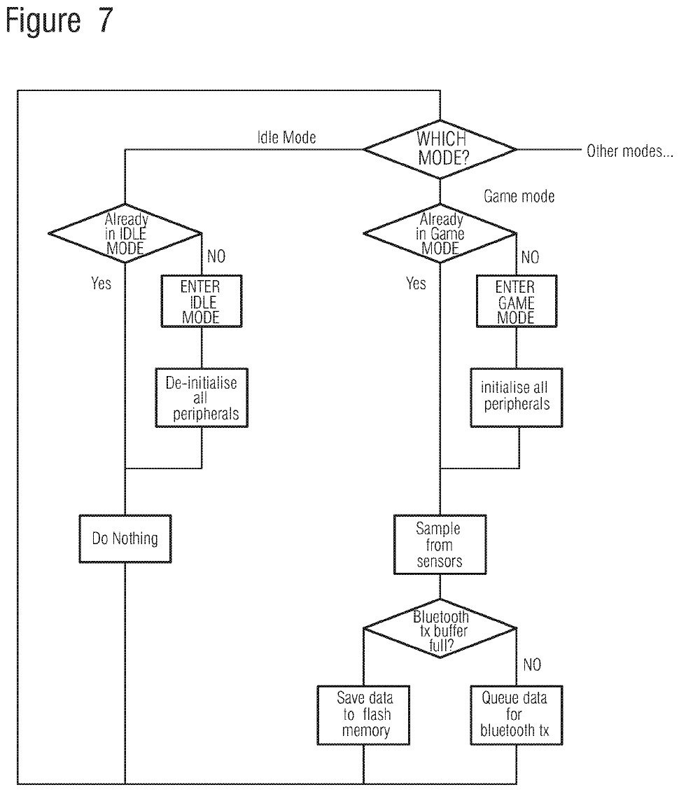

The MCU 21 of the or each mouthpiece 10 runs embedded firmware configured to sample the sensors (PPG, IMU, HIA), save the data to the onboard flash memory module 30 and send that data to the edge device 12. Each mouthpiece (e.g. sports mouthguard) have at least a “game” (or data collection) mode/state, for sampling and logging data during use, and an “idle” mode/state, where nothing is done and power saving is optimised. Referring to FIG. 7 of the drawings, a flowchart of the firmware configuration is provided as an example only. Determination of the required mode of operation may be by means of a user pressing a button (con/off′ button). Alternatively, changing between modes may occur as a result of movement of the mouthpiece (or removal thereof from a charging station) after a period of inactivity (switch to “game” mode) or as a result of a predetermined period of inactivity and/or ‘docking’ the mouthpiece for charging (switch to “idle” mode).

In an alternative exemplary embodiment, the PCB 20 may be mounted in or on a retainer, such as those commonly used for teeth whitening, teeth straightening or protection. The much thinner form factor of such mouthpieces means that the breathing and speech of a user is not significantly impaired, at they can be worn during any activity, whether contact sport or otherwise. The PCB could, in this case, be sandwiched between two layers of material, such as EVA plastic, in a similar manner to that described above. Both layers could be transparent, although the outer layer could, in theory, be formed of a darker material if desired. The retainer thus provides a more streamlined mouthguard, whilst still providing the functionality of the sensor(s) as described above. In this case, a mouthguard which is overmolded to fit over the retainer and the user's teeth can be worn over the retainer when the user participates in contact sports.

It will be apparent to a person skilled in the art, from the foregoing description, that modifications and variations can be made to the described embodiments without departing from the scope of the invention as defined by the appended claims, In the above detailed description, a sports mouthguard is specifically referenced and described. However, it will be appreciated that the invention can be adapted to provide the PPG measurement capability in other types of mouth-worn devices, including dental or orthodontic retainers used for various purposes, and the present invention is not necessarily intended to be limited in this regard.

1. A mouthpiece for a fitness tracking and/or monitoring system, the mouthpiece comprising
a retainer member shaped and configured to be worn, in use, within a user's mouth, the retainer member being formed of at least two layers of elastomeric material having a physiological sensor therebetween.

2. The mouthpiece according to claim 1, wherein said physiological sensor is configured to generate signals representative of a cardio-respiratory signal of a user.

3. The mouthpiece according to claim 2, wherein said physiological sensor comprises a photoplethysmography sensor comprising a light source for, in use, shining a light on a region of the inside of a user's mouth and a sensor for receiving light reflected from said region of the inside of the user's mouth and generating electrical signals representative thereof.

4. The mouthpiece according to claim 1, wherein at least one of the at least two layers of elastomeric material nearest the inner surface of the user's mouth is substantially transparent.

5. The mouthpiece according to claim 1, wherein at least one of the two layers of elastomeric material furthest from the inner surface of the user's mouth is substantially opaque.

6. The mouthpiece according to claim 1, wherein the elastomeric material is an elastomeric polymer, and wherein said elastomeric polymer is Ethylene-Vinyl Acetate.

7. (canceled)

8. The mouthpiece according to claim 1, wherein said physiological sensor is located within said retainer member such that, in use, it is located adjacent the user's palate.

9. The mouthpiece according to claim 1, wherein said physiological sensor is incorporated on a substrate, said substrate further incorporating a control module, a power supply unit and a memory module, and wherein said substrate further incorporates a wireless communication module and antenna configured to wirelessly transmit data representative of signals generated by said sensor to a remote receiver.

10. (canceled)

11. The mouthpiece according to claim 1, wherein said physiological sensor is incorporated at one end of a first elongate flexible connector, and wherein a second elongate flexible connector is provided or integrally formed at the other end of the first elongate flexible connector, said second elongate flexible connector having incorporated at one end thereof at least one or more of a control module, a power supply unit, a memory module, a wireless communication module and a wireless communication antenna.

12. (canceled)

13. The mouthpiece according to claim 11, wherein said first and second elongate flexible connectors, together, form a substantially T-shaped connector.

14. The mouthpiece according to claim 1, wherein multiple physiological sensors are provided on a flexible connector between said first and second layers of elastomeric material, wherein said multiple sensors comprise or include a heart rate monitor and an accelerometer, and wherein said heart rate monitor comprises a photoplethysmograph sensor.

15. (canceled)

16. (canceled)

17. The mouthpiece according to claim 1, wherein the retainer member is formed of at least two layers of elastomeric material, with said physiological sensor being located between said at least two layers, and wherein the mouthpiece further comprises a removable mouthguard or gumshield device configured to fit over the retainer and a user's teeth and gum, in use.

18. (canceled)

19. The mouthpiece according to claim 1, wherein the retainer member is formed of at least three layers of elastomeric material, with said physiological sensor being located between first and second layers, nearest the inner surface of the user's mouth when in use, and a third layer formed over said second layer, wherein said sensor comprises a photoplethysmograph sensor, said first and third layers are substantially transparent and said second layer is substantially opaque, and wherein said photoplethysmograph sensor is provided on a flexible connector and located within the retainer member such that, in use, it is positioned adjacent the user's palate.

20. (canceled)

21. (canceled)

22. A mouthpiece assembly comprising the mouthpiece according to claim 1, and a storage case for receiving said mouthpiece, when not in use, the mouthpiece comprising a receiving element of a wireless charging system and said storage case comprising a transmitting element of a wireless charging system and, wherein said transmitting element is positioned adjacent said receiving element when the mouthpiece is located within the storage case.

23. The mouthpiece assembly according to claim 22, wherein said receiving element comprises a conductive coil printed or otherwise provided on a flexible printed circuit board located between said at least two layers of elastomeric material, and said transmitting element comprises a plurality of overlapping conductive coils.

24. A fitness tracking and/or monitoring system comprising at least one mouthpiece according to claim 1, a receiver for receiving physiological data from the physiological sensor of said at least one mouthpiece and an analytics platform for analysing said physiological data and providing an output indicative of fitness and/or performance of the or each user.

25. A method of manufacturing a mouthpiece for a fitness tracking and/or monitoring system, the method comprising
forming a positive mould of or representative of a user's teeth and gum,
forming a first layer of elastomeric material over said mould,
securing a physiological sensor circuit to said first layer of elastomeric material, and
forming a second layer of elastomeric material over said physiological sensor circuit.

26. The method of manufacturing the mouthpiece according to claim 25, wherein said first layer is substantially transparent, said second layer is substantially opaque, and said sensor circuit comprises a light source, a photoplethysmograph sensor, and a wireless communications module for wirelessly transmitting data representative of signals generated by said sensor to a remote receiver, in use.

27. The method of manufacturing the mouthpiece according to claim 26, wherein said first layer and second layers are substantially transparent, and said sensor circuit comprises a light source, a photoplethysmograph sensor, and a wireless communications module for wirelessly transmitting data representative of signals generated by said sensor to a remote receiver, in use, wherein the method further comprises forming a positive mould of or representative of a user's teeth and gum including the mouthpiece, and forming a mouthguard device over said mould.

28. (canceled)

29. The method of manufacturing the mouthpiece according to claim 27, further comprising forming a third, substantially transparent layer of elastomeric material over said second layer.
